(19) United States
(12) Patent Application Publication
(10) Pub. No.: US 2008/0033053 A1
(43) Pub. Date: Feb. 7, 2008

(54) Cross-Reference To Related Applications

(76) Inventors: Curt D. Wolfgang, Germantown, MD (US); Mihael H. Polymeropoulos, Potomac, MD (US); Christian N. Lavedan, Potomac, MD (US); Simona Volpi, Derwood, MD (US)

Correspondence Address: HOFFMAN WARNICK & D'ALESSANDRO, LLC, 75 STATE STREET, 14TH FLOOR, ALBANY, NY, 12207, US

(21) Appl. No.: 11/572,347

(22) PCT Filed: Jul. 22, 2005
(86) PCT No.: PCT/US05/26050

§ 371 Date: Jan. 19, 2007

Related U.S. Application Data
(60) Provisional application No. 60/590,260, filed on Jul. 22, 2004.

(51) Int. Cl. A61K 31/13; A61P 27/00

(52) U.S. Cl. 514/662

(57) ABSTRACT

Adamantane and other agents with similar effects on gene expression are useful in the treatment or prevention of ocular disorders.

### CROSS-REFERENCE TO RELATED APPLICATIONS

This application claims the benefit of co-pending U.S. Provisional Application No. 60/590,260, filed Jul. 22, 2004, which is hereby incorporated herein.

### BACKGROUND OF THE INVENTION

(1) Technical Field

The present invention relates generally to the treatment of ocular disease and more specifically to protection of retinal nerve fiber function and maintenance of retinal vasculature.

(2) Background of the Invention

Amantadine hydrochloride, i.e., 1-amino adamantane HCl, also known as Symmetrel®, is currently marketed as an antiviral and anti-Parkinson drug. The mechanism of action of amantadine in the treatment of Parkinson's disease is unknown. In addition, a small open-label study in eight patients with Huntington's disease reported a significant reduction of dyskinesias in those patients treated with amantadine. This data may suggest that amantadine may be a potential therapy for Huntington's disease.

The use of other adamantane derivatives in the treatment of certain ocular disorders and disease has also been described.

### SUMMARY OF THE INVENTION

This invention relates to the use of adamantane and derivatives thereof to treat various ocular diseases. In particular, this invention comprises the use of adamantane and derivatives thereof to treat or prevent loss of optic nerve fiber function and for maintenance/restoration of retinal vasculature.

In other aspects, this invention relates to use of agents that are known or found to upregulate certain genes expressed in the eye, i.e., to increase the transcription of certain genes in the eye and/or translation of the RNA transcripts corresponding to those genes. The specific genes are described hereinbelow. In addition to adamantane and derivatives thereof, this invention contemplates the use of other agents that similarly affect gene expression with respect to some or all of the genes described hereinbelow.

Specific diseases in which the use of adamantane or derivative thereof, or of other agents that similarly affect gene expression, will be beneficial include retinal dystrophy, retinal edema, retinal neovascularization, diabetic retinopathy, ischemic retinopathy, vitreoretinopathy, macular edema, age-related macular degeneration, diabetic macular edema, IOP, ocular hypertension, retinitis pigmentosa, choroidal sclerosis, rod/cone degeneration and glaucoma.

A particular aspect of the invention provides a method for treating or preventing at least one ocular disorder selected from the group consisting of: loss of optic nerve fiber, breakdown of retinal vasculature, retinal damage, retinal neovascularization, retinitis pigmentosa, choroidal sclerosis, aged-related macular degeneration, and rod/cone degeneration, the method comprising: internally administering to a patient in need thereof an effective amount of amantadine.

Another aspect of the invention provides a method of protecting against loss of optic nerve fiber function that comprises administering an effective amount of an agent that upregulates expression of at least one of: the CRX gene, a caveolin gene, a crystallin gene, the AKT1 gene, the HSP1A gene, the SLC6A6 gene, and an Aquaporin gene.

A further aspect of the invention provides a method of protecting a patient from retinal damage, such as but not limited to retinal damage resulting from elevated intra-ocular pressure (IOP), comprising: administering an effective amount of an agent that upregulates expression of at least one of: the MYOC gene, the SLC1A3 gene, the IGFBP2 gene, the ASS gene, a crystalline gene, the SLC6A6 gene, an Aquaporin gene, and the GAD1 gene.

Yet another aspect of the invention provides a method of protecting a patient from retinal vascularization comprising: administering an effective amount of an agent that upregulates gene expression of at least one of TIMP3 and TIMP2.

A further aspect of the invention provides a method of identifying drug development candidates for development as retinal neuroprotective agents that comprises comparing the gene expression profile of an untreated test animal with the gene expression profile of an animal treated with a test substance, wherein the test substance is considered a candidate for development as a retinal neuroprotective agent if it is associated with the upregulation of at least one gene selected from a group consisting of CRX, crystallin genes, caveolin genes, AKT1, SLC6A6, MYOC, SLC1A3, ASS, IGFBP2, TIMP3, and Aquaporin genes.

Still another aspect of the invention provides a method of identifying drug development candidates for development as retinal neuroprotective agents that comprises comparing the gene expression profile of an untreated test animal with the gene expression profile of an animal treated with a test substance, wherein the test substance is considered a candidate for development as a retinal neuroprotective agent if it is associated with the downregulation of at least one gene selected from a group consisting of PDCD8, TRADD, and ASNS.

A further aspect of the invention provides a method of maintaining retinal vasculature comprising: administering an effective amount of an agent that upregulates protein expression of at least one of: the CRX gene, a caveolin gene, a crystalline gene, the AKT1 gene, the HSP1A gene, the SLC6A6 gene, and an Aquaporin gene.

A further aspect of the invention provides a method of protecting a patient from retinal damage comprising: administering an effective amount of an agent that upregulates protein expression of at least one of: the MYOC gene, the SLC1A3 gene, the IGFBP2 gene, the ASS gene, a crystallin gene, the SLC6A6 gene, and an Aquaporin gene.

Still a further aspect of the invention provides a method of protecting a patient from retinal vascularization comprising: administering an effective amount of an agent that upregulates protein expression of at least one of the TIMP2 gene and the TIMP3 gene.

Yet another aspect of the invention provides a method of identifying drug development candidates for development as retinal neuroprotective agents comprising: comparing a protein expression profile of an untreated test animal with a protein expression profile of an animal treated with a test substance, wherein the test substance is considered a candidate for development as a retinal neuroprotective agent if it is associated with the upregulation of at least one protein selected from a group consisting of: a CRX protein, a crystallin protein, a caveolin protein, an AKT1 protein, an SLC6A6 protein, an MYOC protein, an SLC1A3 protein, an ASS protein, an IGFBP2 protein, a TIMP3 protein, and an Aquaporin protein.

Another aspect of the invention provides a method of identifying drug development candidates for development as retinal neuroprotective agents comprising: comparing a protein expression profile of an untreated test animal with a protein expression profile of an animal treated with a test substance, wherein the test substance is considered a candidate for development as a retinal neuroprotective agent if it is associated with the downregulation of at least one protein selected from a group consisting of: a PDCD8 protein, a TRADD protein, and an ASNS protein.

Still a further aspect of the invention provides a method for obtaining regulatory approval of a therapeutic agent for treatment or prevention of an ocular disorder comprising: providing to the governmental regulatory agency data demonstrating that the agent at least one of: upregulates expression of at least one of: the CRX gene, a caveolin gene, a crystallin gene, the AKT1 gene, the HSP1A gene, the SLC6A6 gene, and an Aquaporin gene; downregulates expression of at least one of: the PDCD8 gene and the TRADD gene; upregulates expression of at least one of the MYOC gene, the SLC1A3 gene, the IGFBP2 gene, the ASS gene, a crystallin gene, the SLC6A6 gene, an Aquaporin gene, and the GAD1 gene; downregulates expression of the ASNS gene; and upregulates expression of at least one of the TIMP3 gene and the TIMP2 gene.

A further aspect of the invention provides a method of protecting a patient from at least one of: laser treatment and retinal ischemia damage comprising: administering an effective amount of an agent that upregulates expression of at least one of: the TIMP3 gene, the TIMP2 gene, the SULF1 gene, the IRF1 gene, the RBP1 gene, the RBP4 gene, the F3 gene, the CD44 gene, the IRF1 gene, the PLA2G4A gene, and the VEGFB gene.

A still further aspect of the invention provides a method of protecting a patient from at least one of: light and a genetic predisposition damage comprising: administering an effective amount of an agent that upregulates expression of at least one of: the LRAT gene, the RBP1/CRABP-1 gene, the RBP4 gene, the RPE65 gene, and the TTR gene.

The foregoing and other features of the invention will be apparent from the following more particular description of embodiments of the invention.

### DETAILED DESCRIPTION OF THE INVENTION

Adamantane derivatives that are useful in the practice of the present invention include compounds having the core structure of adamantane (tricyclodecane), e.g., memantine, amantadine, and rimantadine. In all cases, useful compounds include salts, stereoisomers, polymorphs, esters, prodrugs, and hydrates and other solvates of adamantane and adamantane derivatives. The preferred compound is amantadine, e.g., amantadine HCl. It has now been found that such agents can be used to treat, i.e., to prevent or treat, ocular disorders as described hereinbelow.

An effective amount of the active agent of the inventions, i.e., adamantane or an adamantane derivative or another agent that has a similar effect on gene expression as described hereinbelow, may be administered to a subject animal (typically a human but other animals, e.g., farm animals, pets, and racing animals, can also be treated) by a number of routes. These include systemic routes of administration, e.g., oral, inhalation, topical, transmucosal, parenteral, intravenous, etc., as well as routes that are intended to provide greater localized administration, e.g., intraocular, intravitreal, intrachoroidal, and topical administration to the eye.

Formulation of the active agent of the invention can be accomplished by routine pharmaceutical formulation techniques depending, e.g., upon the route of administration. The agent can be delivered in immediate release, controlled release, or sustained release forms.

The optimal amount of the active agent to be delivered can be determined by standard techniques. For guidance on routes of administration, formulations and doses for adamantane and derivatives thereof, practitioners can refer to the labeling and other publications relating to Symmetrel® as well as to other publications relating to administration of adamantane and adamantane derivatives for other purposes including those cited herein.

To identify agents other than adamantane that are useful in the practice of the invention, one can set up gene expression assays according to standard techniques. Using such assays, one can readily determine whether or not a compound or other agent, which can include pharmaceutical agents approved for other uses as well as new chemical entities or biopharmaceuticals, which agents have the desired effect on gene expression in the eye.

Before a therapeutic agent (which term includes prophylactic agents) can be commercialized for a given indication, it must be approved by governmental regulatory authorities such as the U.S. Food and Drug Administration and the European Medicines Evaluation Agency. Approval generally requires the submission of data demonstrating the safety and efficacy of the agent. Such data may include gene expression profile data.

Amantadine hydrochloride, also known as Symmetrel®, is currently marketed as an antiviral and anti-Parkinson drug. While amantadine has been shown to have many biological actions, especially in neurons and in the brain, the molecular mechanisms behind these biological activities remain elusive. Therefore, in order to identify the molecular pathways regulated by amantadine, Sprague Dawley rats were treated with different doses of amantadine and RNA expression profiling analysis was performed on selected tissues. This report describes results obtained from the analysis of the retina from those animals sacrificed at steady state. The changes in gene expression suggest that amantadine influences expression of genes that may result in a neuroprotection. Therefore, these data indicate that amantadine could be used to protect against retinal ganglion cell loss in diabetic retinopathy, diabetic macular edema, aged-related macular degeneration, glaucoma and rod/cone loss in retinitis pigmentosa, rod/cone dystrophies and choroidal sclerosis.

Amantadine is freely soluble in water and is well absorbed (Endo). Amantadine is primarily excreted unchanged in the urine by glomerular filtration and renal tubular secretion (Endo; Goralski, Smyth, and Sitar 496-504). In humans, the time to reach peak concentration (Cmax) is 3.3±1.5 hours (range: 1.5-8 hours) and the half-life is 17±4 hours (range: 10-25 hours) (Endo). Amantadine has been reported to be teratogenic in rats at 50 mg/kg/day and embryotoxic at 100 mg/kg/day (estimated human equivalent dose (HED) of 7.1 mg/kg/day and 14.2 mg/kg/day, respectively, based on body surface area conversion) (Endo). A dose of 37 mg/kg/day (estimated HED 5.3 mg/kg/day) did not produce teratogenic or embryotoxic effects in the rat (Endo). While long-term in vivo animal studies to evaluate the carcinogenic potential of amantadine have not been performed, amantadine has been shown to be non-mutagenic in the Ames Test or in Chinese Hamster Ovary cells (Endo). Furthermore, no evidence of chromosomal damage was observed in vitro in human peripheral blood lymphocytes or in an in vivo mouse bone marrow micronucleus test (Endo).

While amantadine has been shown to have many biological actions, especially in neurons and in the brain, the molecular mechanisms behind these biological activities still remain elusive. Therefore, in order to identify the molecular pathways regulated by amantadine, Sprague Dawley rats were treated with different doses of amantadine for different time periods: 3 hours (Cmax), 14 days (Steady State), and 14 days followed by 3 days with no treatment (Recovery). The animals were sacrificed at the appropriate times and their tissues were collected for RNA expression profiling analysis. The analysis of gene expression profiles influenced by amantadine treatment not only sheds light on its mechanism of action, but also identifies new therapeutic indications for this drug. Gene expression profiles include measurements of proteins and/or transcripts. This report describes results obtained from the analysis of the retina from those animals sacrificed at steady state. The changes in gene expression suggest that amantadine influences expression of genes that may result in a neuroprotection. Therefore, these data indicate that amantadine is useful to protect against retinal ganglion cell loss in diabetic retinopathy, diabetic macular edema, age-related macular degeneration, glaucoma and rod/cone loss in retinitis pigmentosa, rod/cone dystrophies and choroidal sclerosis.

1. Materials and Methods

1.1 Animal Treatment Protocol

All animal services were outsourced to Charles River Laboratories, under Study Number 231-001. Thirty male Sprague Dawley out-bred albino rats (Crl: CD® (SD) IGS BR) were used in this study and received from Charles River Laboratories, Inc. (Raleigh, N.C.). The animals were acclimated for eight days prior to Study Day 1 and were examined by the Staff Veterinarian prior to being released for use on the study. The rats were randomly assigned to groups by a computer generated weight-ordered distribution such that group mean body weights did not exceed ±10% of the overall mean weight. On Study Day 1, the animals were approximately 11 weeks-old and weighed 300-369 grams. The study design is shown in Table 1.

TABLE 1
VFS-947 Study Design

| | | Dosage | Dosage | Dosage | Number |
|---|---|---|---|---|---|
| Group | Group | Level | Concentration | Volume | of |
| No. | Designation | (mg/kg) | (mg/mL) | (mL/kg) | Males |
| 1 | Untreated^{a} | NA | NA | NA | 3 |
| 2 | Vehicle | 0 | 0 | 5 | 9 |
| | Control | | | | |
| | (dH₂O) | | | | |
| 3 | Low-dose | 20^{b} | 4 | 5 | 9 |
| 4 | High-Dose | 100^{c} | 20 | 5 | 9 |

^{a}Animals in Group 1 were not treated with vehicle control or test article.
^{b}This dosage was based on the HED using the formula [HED = animal dose * 0.16].
^{c}This dosage was designed to be five times the HED.

Doses were administered once daily via intraperitoneal injection to animals in Groups 2, 3 and 4. Animals in Group 1 were untreated. The animals in Group 2 were treated with the vehicle control (dH₂O) each day for up to 14 consecutive days. The animals in Group 3 and 4 were treated with the test article each day for up to 14 consecutive days. On Study Day 1 at three hours postdose (Tmax), three animals/group in Groups 2, 3 and 4 were euthanized along with the three untreated animals in Group 1. On Study Day 14 (Steady State), at three hours postdose, three animals per group in Groups 2, 3 and 4 were euthanized. Following a three-day washout period, the remaining animals in Groups 2, 3, and 4 were euthanized on Study Day 17 (recovery). Euthanasia was performed via decapitation without anesthesia in accordance with accepted American Veterinary Association guidelines.

After euthanasia, retinas were collected and snap frozen in liquid nitrogen. All samples were shipped to Vanda Pharmaceuticals on dry ice and were stored at −80° C. until use.

1.2 RNA Extraction

RNA was extracted according to standard RNA extraction protocols and RNA quantification was performed using a spectrophotometer.

1.3 RNA Expression Profiling

RNA expression profiling was performed using the Rat Expression Array 230A and 230 v 2.0 following the manufacturer's standard protocol (Affymetrix, Santa Clara, Calif.).

1.4 Gene Expression Analysis

We identified genes that were differentially expressed as a result of arrays derived from retinas treated with vehicle, “baseline chips,” and arrays derived from retinas treated with amantadine hydrochloride (low dose [LD] or high dose [HD]), “test chips.” Two types of analyses were performed using a filter of p<0.05 and a fold change of 1.5 or 1.6, with all absent probe sets being excluded from the analysis: (1) vehicle versus low dose and (2) vehicle versus low+high doses.

2. Results

2.1 RNA Expression Analysis

2.1.1 Retina: Vehicle vs. Low Dose at Steady State

As a first step towards understanding the biological actions of amantadine, we compared the RNA expression profiles of retinas from rats treated with vehicle (Group 2) to rats treated with the low dose of amantadine (Group 3, 20 mg/kg/day) at steady state. The rationale behind this approach is three-fold. First, the 20 mg/kg/day dose is representative of the daily prescribed HED (human equivalent dose). Second, to identify a possible new indication, it is important to know the changes in gene expression after constant long-term exposure of the drug. While most changes in gene expression happen immediately after exposure (i.e. at the Tmax/3 hour timepoint), these changes may be considered more as a “transient” adaptation response to drug treatment. Third, amantadine is well documented to have a biological function in the brain, while nothing is known about its potential action in the retina. In addition, the retina is a relatively “clean” tissue in the sense that when extracted from the rat, one can be confident that it is not contaminated by another tissue/structure.

A comparison analysis was performed to identify genes whose expression changed ≧1.6 or 1.5 fold (either up- or down-regulated) between the two treatment groups and was statistically significant (p<0.05, T-test). Analysis of the probe sets identified many groups of genes encoding proteins that have a similar biological function. For example, amantadine altered the expression of many solute/ion-channel proteins (KCNE2, SLC1A3, SLC3 A1, SLC4A3, SLC6A6, SLC7A1, SLC7A8, SLC17A7, SLC21A5, SLC24A1 and SLC26A1), proteins directly or indirectly involved in glutamate synthesis (ASNS, ASS, GAD1), proteins involved in maintenance of cell-cell interactions (TIMP2, TIMP3, SERPINI1), lens structural proteins (CRYAB and CRYBA3) and apoptosis (PDCD8).

The overall theme of the gene list sindicates that amantadine plays role in regulating genes involved in neuroprotection (cell cycle and apoptosis), the retinoid cycle, the coagulation pathway, and angiogenesis. The significance of these findings is elaborated in the discussion.

TABLE 2
Genes involved in neuroprotection (VEH vs. LD)

| Affy Probe | Fold | P-Value | UniGene | Gene | |
|---|---|---|---|---|---|
| Set | Change | (t-test) | Link | Symbol | Gene Description |
| 1370026_at | 4.15 | 0.016642 | Rn.98208 | CRYAB | Crystallin, alpha B |
| 1368440_at | 3.51 | 0.003900 | Rn.11196 | SLC3A1 | Solute carrier family 3, member 1 |
| 1368987_at | 3.32 | 0.012316 | Rn.10267 | SCL17A7 | Solute carrier family 17 (sodium- |
| | | | | | dependent inorganic phosphate |
| | | | | | cotransporter), member 7 |
| 1388064_a_at | 3.23 | 0.000164 | Rn.34134 | SLC1A3 | Solute carrier family 1, member 3 |
| 1387313_at | 3.19 | 0.042537 | Rn.30051 | MYOC | Myocilin |
| 1387829_at | 3.10 | 0.024217 | Rn.48143 | SLC24A1 | Sodium/calcium/potassium exchanger |
| 1368778_at | 3.06 | 0.011404 | Rn.9968 | SLC6A6 | Solute carrier family 6, member 6 |
| 1370760_a-at | 2.93 | 0.013559 | Rn.91245 | GAD1 | Glutamate decarboxylase 1 |
| 1370964_at | 2.84 | 0.015220 | Rn.5078 | ASS | Arginosuccinate synthetase |
| 1370101_at | 2.76 | 0.040780 | Rn.44287 | CRX | Cone-rod homeobox protein |
| 1387057_at | 2.30 | 0.047159 | Rn.82734 | SCL7A8 | Solute carrier family 7 (cationic amino |
| | | | | | acid transporter, y+ system), member 8 |
| 1368600_at | 1.86 | 0.011841 | Rn.10016 | SLC26A1 | solute carrier family 26 (sulfate |
| | | | | | transporter), member 1 |
| 1387094_at | 1.83 | 0.044897 | Rn.5641 | SLC21A5 | solute carrier family 21 (organic anion |
| | | | | | transporter), member 5 |
| 1368772_at | 1.73 | 0.04826 | Rn.87739 | SLC4A3 | solute carrier family 4, member 3 |
| 1368391_at | −1.7 | 0.037643 | Rn.9439 | SLC7A1 | solute carrier family 7, member 1 |
| 1370321_at | −1.73 | 0.006841 | Rn.8124 | PDCD8 | programmed cell death 8 (apoptosis- |
| | | | | | inducing factor) |
| 1387925_at | −2.12 | 0.006218 | Rn.11172 | ASNS | Asparagine synthetase |
| 1368247_at | 1.82 | 0.034955 | Rn.1950 | HSPA1A | heat shock 70 kD protein 1A |

TABLE 3
Genes involved in angiogenesis (VEH vs. LD)

| Affy Probe | Fold | P-Value | UniGene | Gene | |
|---|---|---|---|---|---|
| Set | Change | (t-test) | Link | Symbol | Gene Description |
| 1372926_at | 2.34 | 0.000875 | Rn.98839 | TIMP3 | Tissue inhibitor of metalloproteinase 3 |
| 1367823_at | 1.83 | 0.025038 | Rn.10161 | TIMP2 | tissue inhibitor of metalloproteinase 2 |
| 1368187_at | 2.05 | 0.006056 | Rn.13778 | GPNMB | glycoprotein (transmembrane) nmb |
| 1368771_at | 1.51 | 0.008621 | Rn.20664 | SULF1 | sulfatase FP |
| 1368073_at | 1.73 | 0.010855 | Rn.6396 | IRF1 | interferon regulatory factor 1 |
| 1367939_at | 1.89 | 0.034195 | Rn.902 | RBP1 | retinol binding protein 1 |
| 1371762_at | 1.7 | 0.03534 | Rn.3477 | RBP4 | Rattus norvegicus cDNA clone |
| | | | | | MGC: 72936 IMAGE: 6890712, |
| | | | | | complete cds |
| 1380854_at | 1.74 | 0.043142 | | VEGFB | vascular endothelial growth factor B |

TABLE 4
Genes involved in coagulation (VEH vs. LD)

| Affy Probe | Fold | P-Value | UniGene | Gene | |
|---|---|---|---|---|---|
| Set | Change | (t-test) | Link | Symbol | Gene Description |
| 1369182_at | 1.87 | 0.007962 | Rn.9980 | F3 | coagulation factor 3 |
| 1368921_a_at | 1.72 | 0.017761 | Rn.1120 | CD44 | CD44 antigen |
| 1368073_at | 1.71 | 0.025642 | Rn.6396 | IRF1 | interferon regulatory factor 1 |
| 1387566_at | 1.51 | 0.01006 | Rn.10162 | PLA2G4A | phospholipaseA2, group IVA |
| | | | | | (cytosolic, calcium-dependent) |
| 1380854_at | 1.74 | 0.043142 | | VEGFB | vascular endothelial growth factor B |
| 1368349_at | 2.01 | 0.004121 | Rn.6346 | FGFBP1 | growth factor binding protein-1 |

TABLE 5
Genes involved in the retinoid cycle (VEH vs. LD)

| Affy Probe | Fold | P-Value | UniGene | | |
|---|---|---|---|---|---|
| Set | Change | (t-test) | Link | Gene Symbol | Gene Description |
| 1368570_at | 2 | 0.046578 | Rn.54479 | LRAT | lecithin-retinol acyltransferase |
| 1367939_at | 1.89 | 0.034195 | Rn.902 | RBP1/CRABP-1 | retinol binding protein 1 |
| 1371762_at | 1.7 | 0.03534 | Rn.3477 | RBP4 | Rattus norvegicus cDNA clone |
| | | | | | MGC: 72936 IMAGE: 6890712, |
| | | | | | complete cds |
| 1389473_at | 2.7 | 0.046201 | Rn.21866 | | Rattus norvegicus transcribed |
| | | | | | sequence with weak similarity |
| | | | | | to protein sp: P47804 |
| | | | | | (H. sapiens) RGR_HUMAN |
| | | | | | RPE-retinal G protein-coupled |
| | | | | | receptor |
| 1369056_at | 2.52 | 0.009223 | Rn.76724 | RPE65 | retinal pigment epithelium, 65 kDa |
| 1367598_at | 2.21 | 0.009978 | Rn.1404 | TTR | transthyretin |
| 1368437_at | −2.4 | 0.032558 | Rn.9155 | CA4 | carbonic anhydrase 4 |

2.1.2 Retina: Vehicle vs. (Low Dose & High Dose) at Steady State

As a second step towards understanding the biological actions of amantadine, we grouped together the RNA expression profiles of retinas from rats treated with either low dose of amantadine (Group 3, 20 mg/kg/day) or high dose of amantadine (Group 4, 100 mg/kg/day) into one treatment group, and compared them to the RNA expression profiles of retinas from the rats treated with vehicle only. Combining the two amantadine groups provided more statistical power to identify important changes in gene expression, regardless of the dose.

A comparison analysis was performed to identify genes whose expression changed ≧1.6-fold (either up- or down-regulated) between the two treatment groups and was statistically significant (p<0.05, T-test). The analysis of the probe sets identified several groups of genes encoding proteins that have a similar biological function. For example, amantadine altered the expression of multiple lens structural proteins (CRYAA, CRYAB, CRYBA2, CRYBA4, CRYBB3, CRYBS), aquaporins (AQP1, AQP4) solute/ion-channel proteins (CACNB2, KCNE2, SLC1A3, SLC3A1, SLC4A3, SLC6A6, SLC7A1, SLC7A8, SLC17A7, SLC21A5, SLC24 A1, SLC24A2 and SLC26A1), proteins directly or indirectly involved in glutamate synthesis (ASNS, ASS, GAD1, GLYT1), proteins involved in maintenance of cell-cell interactions (TIMP2, TIMP3, SERPINI1), and apoptosis (CAV1, PDCD8, TRADD).

As before, we identified a major theme in the gene list. It appears that the most significant group is centered around CAV1, a scaffolding protein found in the Golgi caveolae plasma membranes that has been implicated in mitogenic signaling and oncogenesis (Fiucci et al. 2365-75) and has been reported have antiapoptotic activities (Li et al. 9389-404). The significance of these findings is elaborated in the discussion.

TABLE 6
Genes involved in neuroprotection (VEH vs. LD + HD)

| Affy Probe | Fold | P-Value | UniGene | Gene | |
|---|---|---|---|---|---|
| Set | Change | (t-test) | Link | Symbol | Gene Description |
| 1367608_at | 21.20 | 0.041862 | Rn.10802 | CRYBA4 | Crystalline, beta A4 |
| 1367990_at | 19.59 | 0.030844 | Rn.19693 | CRYBB3 | Crystalline, beta B3 |
| 1370279_at | 19.07 | 0.012603 | Rn.44585 | CRYAA | Crystalline, alpha A |
| 1367684_at | 18.55 | 0.016544 | Rn.10350 | CRYBB2 | Crystallin, beta B2 |
| 1388385_at | 16.28 | 0.035281 | Rn.19713 | CRYBA2 | betaA2-crystallin |
| 1370026_at | 3.83 | 0.016099 | Rn.98208 | CRYAB | Crystallin, alpha B |
| 1368987_at | 3.44 | 0.031196 | Rn.10267 | SLC17A7 | Solute carrier family 17 (sodium- |
| | | | | | dependent inorganic phosphate |
| | | | | | cotransporter), member 7 |
| 1387829_at | 3.21 | 0.013882 | Rn.48143 | SLC24A1 | Sodium/calcium/potassium exchanger |
| 1368440_at | 3.11 | 0.014221 | Rn.11196 | SLC3A1 | Solute carrier family 3, member 1 |
| 1370760_a_at | 3.03 | 0.003904 | Rn.91245 | GAD1 | Glutamate decarboxylase 1 |
| 1388064_a_at | 2.94 | 0.000237 | Rn.34134 | SLC1A3 | Solute carrier family 1, member 3 |
| 1368778_at | 2.89 | 0.008844 | Rn.9968 | SLC6A6 | Solute carrier family 6, member 6 |
| 1370101_at | 2.65 | 0.010503 | Rn.44287 | CRX | Cone-rod homeobox protein |
| 1387313_at | 2.58 | 0.007217 | Rn.30051 | MYOC | Myocilin |
| 1370964_at | 2.56 | 0.003579 | Rn.5078 | ASS | Arginosuccinate synthetase |
| 1370131_at | 2.42 | 0.047256 | Rn.22518 | CAV | caveolin |
| 1373561_at | 2.40 | 0.005418 | Rn.3794 | | Rattus norvegicus transcribed sequence |
| | | | | | with strong similarity to protein |
| | | | | | ref: NP_078812.1 (H. sapiens) |
| | | | | | hypothetical protein FLJ22578 [Homo |
| | | | | | Sapiens] |
| 1372926_at | 2.38 | 0.006743 | Rn.98839 | TIMP3 | Tissue inhibitor of metalloproteinase 3 |
| 1375468_at | 2.24 | 0.009319 | Rn.19957 | ABCC5A | ATP-binding cassette, sub-family C |
| | | | | | (CFTR/MRP), member 5a |
| 1369625_at | 2.14 | 0.024123 | Rn.1618 | AQP1 | Aquaporin 1 |
| 1367648_at | 2.13 | 0.005536 | Rn.6813 | IGFBP2 | Insulin-like growth factor binding |
| | | | | | protein 2 |
| 1387146_a_at | 2.11 | 0.002624 | Rn.11412 | EDNRB | Endothelin receptor type B |
| 1370135_at | 1.99 | 0.035759 | Rn.81070 | CAV2 | Caveolin 2 |
| 1387397_at | 1.87 | 0.039635 | Rn.90091 | AQP4 | Aquaporin 4 |
| 1368862_at | 1.69 | 0.004759 | Rn.11422 | AKT1 | v-akt murine thymoma viral oncogene |
| | | | | | homology 1 |
| 1387651_at | 1.68 | 0.005841 | Rn.1618 | AQP1 | Aquaporin 1 |
| 1388000_at | 1.66 | 0.043338 | Rn.74242 | SLC24A2 | Solute carrier family 24 (sodium/ |
| | | | | | potassium/calcium exchanger), |
| | | | | | member 2 |
| 1368247_at | 1.64 | 0.022058 | Rn.1950 | HSPA1A | Heat shock 70 kD protein 1A |
| 1370321_at | −1.74 | 0.031477 | Rn.8124 | PDCD8 | Programmed cell death 8 (apoptosis- |
| | | | | | inducing factor) |
| 1368391_at | −1.91 | 0.001898 | Rn.9439 | SLC7A1 | Solute carrier family 7, member 1 |
| 1387925_at | −2.36 | 0.000235 | Rn.11172 | ASNS | Asparagine synthetase |

3. Discussion

Amantadine hydrochloride is currently marketed as an antiviral and anti-Parkinson drug (Endo). The mechanism of action of amantadine is not understood. To investigate the mechanism of action and potentially identify new indications, we treated rats with different doses of amantadine and performed gene expression profiling. The analysis of the retina indicates that amantadine is useful as a neuroprotective agent to prevent retinal ganglion cell loss, as well as an agent to reduce intraocular pressure. Hence, data indicate that amantadine is useful for retinal dystrophy, diabetic retinopathy, diabetic macular edema and glaucoma. The support for these claims is discussed below.

3.1 Neuroprotection

The first gene indicating a neuroprotective role for amantadine is cone-rod homeobox (CRX). CRX is an otd/Otx-like homeodomain transcription factor that is predominantly expressed in the rod and cone of photoreceptors of the retina (Furukawa, Morrow, and Cepko 531-41). CRX binds to and activates the promoters of a number of photoreceptor genes including rhodopsin, β-phosphodiesterase, arrestin, and interphotoreceptor retinoid-binding protein (Chen et al. 1017-30). The importance of CRX was initially identified in a study of mutant mice that are homozygous for a null CRX allele. Mice who lack a functional CRX allele do not develop functional photoreceptor outer segments and undergo retinal degeneration (Furukawa et al. 466-70). Gene expression analyses of these mice revealed reduced or lost expression of many photoreceptor-specific genes before the onset of degeneration, suggesting that CRX is a significant regulator of photoreceptor gene expression (Livesey et al. 301-10). The importance of CRX in retinal function is further supported by the fact that numerous mutations in this gene have been linked to retinal degeneration (Freund et al. 543-53; Jacobson et al. 2417-26; Swain et al. 1329-36). The fact that CRX was found to be up-regulated 2.7 fold in retinas of amantadine-treated animals indicates that amantadine has a neuroprotective effect to promote photoreceptor function and minimize retinal degeneration.

The next family of genes indicating a neuroprotective role for amantadine is the crystallins. Cystallins are a diverse group of proteins that are expressed at high levels in lens fiber cells as well as retinal nuclear layers (Xi et al. 410-19). These proteins have been shown to have chaperone functions; members of the small heat-shock family of proteins that protect other proteins from stress-induced aggregation by recognizing and binding to partially unfolded species of damaged proteins (Schey et al. 200-03). Interestingly, heat shock protein 70 kDa 1A was also induced 1.6 fold by amantadine treatment. Crystallins have also been shown to have anti-apoptotic activities as well by inhibiting the activation of caspases (Mao et al. 512-26; Xi et al. 410-19). The end result would therefore inhibit premature cell death. The importance of crystallins in eye function has been demonstrated also by the identification of mutations in several of the crystallins which lead to progressive, regressive and dominant cataracts (Graw and Loster 1-33). Several crystallins are significantly up-regulated (4-21 fold) in retinas of amantadine-treated rats. Therefore, by inducing the expression of crystallins and heat shock protein 1A, amantadine can protect the retina from cell death by inducing these anti-apoptotic proteins.

Many other genes involved in apoptosis/premature cell death were also found to be differentially expressed upon amantadine treatment. For example, caveolin 1 and caveolin 2 were found to be up-regulated 2.42- and 1.99-fold, respectively. As indicated previously, caveolins have been reported to have anti-apoptotic activities (Li et al. 9389-404).

AKT1 was also up-regulated by amantadine treatment. AKT1 is a serine/threonine kinase that plays a major role in transducing proliferative and survival signals intracellularly (Marte and Downward 355-58). AKT1 has been demonstrated to phosphorylate a number of proteins involved in apoptotic signaling cascades; phosphorylation of these proteins prevents apoptosis and promotes cell survival by several different mechanisms (Trencia et al. 4511-21).

In addition to the caveolins and AKT, EDNRB was upregulated. Endothelin receptor B is associated with neuronal survival in brain. Endothelin, a vasoconstrictive peptide, acts as anti-apoptotic factor (Yagami et al. 291-300). Therefore, the up-regulation of these genes by amantadine would protect the retina from premature cell death.

On the other hand, two genes known to induce apoptosis, namely PDCD8 and TRADD, were found to be down-regulated in retinas following amantadine treatment. PDCD8, also known as apoptosis-inducing factor, is localized to mitochondria and is released in response to death stimuli (Joza et al. 549-54). Genetic inactivation of PDCD8 renders cells resistant to cell death (Joza et al. 549-54). TRADD, a protein that specifically interacts with an intracellular domain of tumor necrosis factor receptor 1, has been shown to be essential for mediating programmed cell death (Hsu, Xiong, and Goeddel 495-504). Hence, the down-regulation of these genes by amantadine would also protect the retina from premature cell death. Therefore, the results presented in this study indicate that amantadine is useful as a neuroprotective agent to protect retinal cells from cell death.

3.2 Intraocular Pressure and Glaucoma

Glaucoma can be defined as a group of optic neuropathies characterized by the death of retinal ganglion cells accompanied by excavation and degeneration of the optic nerve head (Ahmed et al. 1247-58). One major risk factor for the development of glaucoma is elevated intraocular pressure (IOP). In a study to identify gene expression changes in retinas after chronic elevation of IOP, Tomarev and colleagues performed microarray analysis of retinas from rats that experienced elevated IOP for five weeks. Their analysis identified 74 genes that were up-regulated and seven genes that were down-regulated in the retina, in so producing an “elevated IOP gene signature” in the retina. Interestingly, some of the genes they found down-regulated in their study were found to be up-regulated in the amantadine experiment, and vice versa. For example, CRYAB, CRYAA, CRYBB2, and SLC6A6 were found to be down-regulated −5.0, −14.5, −18.0 and −2.1-fold, respectively, in the IOP study, while they were up-regulated 3.83, 19.07, 18.55 and 2.89-fold, respectively, in the amantadine study.

The biological significance of crystallins has previously been described. SLC6A6, also known as the taurine transporter, is involved in neural excitability and osmoregulation. Taurine is a semi-essential amino acid that is not incorporated into proteins and is found in high millimolar concentrations in the retina (Militante and Lombardini 75-90; Schuller-Levis and Park 195-202). It has been established that visual dysfunction and retinal lesions results from taurine deficiency (Militante and Lombardini 75-90). In addition, mice with the taurine transporter knocked out show vision loss due to severe apoptotic retinal degeneration (Schuller-Levis and Park 195-202). Importantly, amantadine treatment caused the upregulation of the taurine transporter in the retina. These data indicate that amantadine is useful as a protective agent against retinal damage caused by elevations in IOP.

Glucocorticoid eye drops, commonly in the form of dexamethasone, are commonly used to treat eye inflammation. Dexamethasone is known to cause a form of open-angle glaucoma that involves increased resistance to aqueous humor outflow through the trabecular meshwork (TM) (Ishibashi et al. 3691-97). The prolonged effects of dexamethasone treatment on TM cells identified the first glaucoma gene, namely myocilin (MYOC) (Leung et al. 425-39). MYOC mutations have recently been shown to cause glaucoma (Alward et al. 1022-27; Fingert et al. 899-905; Stone et al. 668-70). Interestingly, MYOC was found to be up-regulated 2.58-fold in retinas from rats treated with amantadine. To identify genes related to the occurrence of steroid-induced glaucoma, two groups independently performed gene expression analysis studies on cultured TM cells treated with dexamethasone. Both studies identified MYOC and insulin-like growth factor binding protein 2 (IGFBP2) to be up-regulated and asparagines synthetase to be down-regulated by dexamethasone treatment (Ishibashi et al. 3691-97; Leung et al. 425-39). Similar regulation of these genes was identified in retinas from rats treated with amantadine. It is unclear at this time whether the changes of gene expression are a protective effect against the damage caused by dexamethasone or a result of the damage. Further studies are needed to clarify this point. However, if these changes were to be protective, this finding would strengthen the hypothesis that amantadine is useful as a protective agent against retinal damage caused by elevations in IOP.

Aquaporins are water transporting proteins and play a role in many aspects of eye function that involve fluid transport across membranous barriers, such as regulation of IOP and retinal signal transduction (Verkman 137-43). Both aquaporin 1 and 4 (AQP1 and AQP4) were found to be up-regulated after amantadine treatment. AQP4 has been shown to be important in retinal signal transduction and AQP1 has been found to be involved in the maintenance of TM cells (Verkman 137-43). The upregulation of these genes by amantadine further indicates a therapeutic role for amantadine for treating increased IOP.

Glutamate is the principal excitatory neurotransmitter in the mammalian central nervous system and excessive levels of glutamate have been implicated in the pathogenesis of glaucoma (Naskar, Vorwerk, and Dreyer 1940-44). Under normal conditions, glutamate transporters rapidly transport glutamate into the intracellular space to maintain physiological concentrations in the eye (Nicholls and Attwell 462-68). To date, five excitatory amino acid transporters (EAAT1-5) have been identified to be involved in the clearance of glutamate in the nervous system. Specifically, EAAT1 is found in the retina (Rauen, Rothstein, and Wassle 325-36). The expression of this glutamate transporter has been found to be reduced in glaucoma (Naskar, Vorwerk, and Dreyer 1940-44). Importantly, this transporter (also known as SLC1A3) was found to be up-regulated in retina from animals treated with amantadine. The upregulation of this gene would result in more transporter expression and less glutamate found within the vitreous humor.

Along with the transporter, other genes involved in glutamine synthesis were also found to be differentially expressed after amantadine treatment. Specifically, asparagine synthetase (ASNS) was found to be down-regulated after amantadine treatment. ASNS is involved in the catalysis of two biochemical reactions: (1) conversion of aspartate to Asparagine, and (2) conversion of aspartate and glutamine to asparagine and glutamate. Having less ASNS expressed would result in less glutamate protection, thereby relieving the retina from the toxicity of excess glutamate. In addition to ASNS, Arginosuccinate synthetase (ASS) was found to be up-regulated after amantadine treatment. ASS is involved in the conversion of aspartate to arginine, which would have an indirect effect on the amount of glutamate that is produced. By increasing the amount of ASS expression, the available aspartate would be converted to arginine, thereby decreasing the amount available to be converted to glutamate.

In addition, amantadine down-regulates CA4, a member of the family of carbonic anhydrases (CAs). CA4 is functionally important in CO₂ and bicarbonate transport; it is membrane-bound enzyme located in the extracellular part of the corneal endothelium. A key event in glaucoma is the catalytic formation of HCO₃₋ from CO₂ and OH. Therefore, amantadine by decreasing CA4 expression could inhibit HCO₃₋ synthesis which in turn would reduce aqueous formation and lowers pressure in glaucoma patients (Maren, 1976; id). Therefore, the results shown clearly demonstrate the possibility of amantadine being used in the treatment of elevated intraocular pressure for the prevention of retinal degeneration.

3.3 Diabetic Retinopathy and Diabetic Macular Edema

Diabetic retinopathy and diabetic macular edema are common microvascular complications in patients with diabetes and may have a sudden and debilitating impact on visual acuity, eventually leading to blindness (Ciulla, Amador, and Zinman 2653-64). In developed countries, diabetic retinopathy is recognized as the leading cause of blindness in the working-age population (20-74 years old) and is responsible for 12% of new cases of blindness each year (Ciulla, Amador, and Zinman 2653-64). Over a 10-year period, diabetic macular edema will develop in 10-14% of Americans with diabetes (Klein, Klein, and Moss 796-801). Diabetic retinopathy and diabetic macular edema is characterized by the growth of abnormal retinal blood vessels which leads to retinal thickening in the macular area and breakdown of the blood-retinal barrier because of leakage of dilated hyperpermeable capillaries and microaneurysms (Ciulla, Amador, and Zinman 2653-64). Breakdown of the inner blood-retinal barrier results in the accumulation of extracellular fluid in the macula, which eventually leads to elevated IOP (Antcliff and Marshall 223-32). In addition, hyperglycemia of diabetes leads to the buildup of intracellular sorbitol and fructose in the retina (Gabbay 521-36). The ensuing disruption of the osmotic balance of the retina is believed to result in cellular damage, which may be important in the loss of integrity of the blood-retinal barrier, among other complications (Gabbay 521-36).

As described previously, amantadine induces genes involved in protecting cells from premature cell death, as well as inducing the expression of the aquaporins, the taurine transporter, and many other solute carrier transport channels which are involved in maintaining osmotic homeostasis in the eye. The up-regulation of these genes will therefore help protect the retina from the damage caused by diabetic retinopathy and diabetic macular edema, thereby supporting the use of amantadine as a therapeutic for diabetic retinopathy and diabetic macular edema.

3.4 Age-Related Macular Degeneration

Macular degeneration is a retinal degenerative disease that causes progressive loss of central vision by the degeneration of the macula. The risk of developing macular degeneration increases with age. The macula is the central portion of the retina responsible for perceiving fine visual detail. Light sensing cells in the macula, known as photoreceptors, convert light into electrical impulses and then transfer these impulses to the brain via the optic nerve.

There are two types of Macular Degeneration: dry and wet. Dry macular degeneration accounts for about 90 percent of all cases. It is sometimes called atrophic, nonexudative, or drusenoid macular degeneration. With dry macular degeneration, yellow-white deposits called Drusen accumulate in the retinal pigment epithelium (RPE) tissue beneath the macula. Drusen deposits are composed of waste products from photoreceptor cells. For unknown reasons, RPE tissue can lose its ability to process waste. As a result, Drusen deposits accumulate. These deposits are thought to interfere with the function of photoreceptors in the macula, causing progressive degeneration of these cells.

Wet macular degeneration instead accounts for about 10 percent of cases. Wet macular degeneration is also called choroidal neovascularization, subretinal neovascularization, exudative, or disciform degeneration. In wet macular degeneration, abnormal blood vessel growth forms beneath the macula. These vessels leak blood and fluid into the macula damaging photoreceptor cells. Wet macular degeneration tends to progress rapidly and can cause severe damage to central vision (information provided by Foundation Fighting Blindness at http://www.blindness.org/).

Recently, there has been considerable progress in developing treatments for macular degeneration. Laser photocoagulation, in some cases of wet macular degeneration (macular degeneration extra-foveal CNV-choroidal neovascularization) is the preferred treatment method.

As described above, amantadine up-regulates the expression of several genes involved in the coagulation pathway (CD44, F3, IRF1, PLA2G4A, and VEGF).

CD44 antigen together with VEGF have been shown to be maximally induced at 3-5 days post laser photocoagulation, and were localized to RPE, choroidal vascular endothelial and inflammatory cells (Shen et al. 1063-71).

F3 (tissue factor) is known to be involved in the coagulation cascade. F3 is usually released when the activation of the extrinsic pathway is initiated upon vascular injury and is a cofactor in the factor VIIa-catalyzed activation of factor X (Frederick et al. 397-417). PLA2G4A (Cytosolic phospholipase A2) catalyzes the release of arachidonic acid from membrane phospholipids. Arachidonic acid in turn serves as precursor for a wide spectrum of biologic effectors, collectively known as eicosanoids that are involved in hemodynamic regulation, inflammatory responses, and other cellular processes. The arachidonic acid release leads to an increase in thromboxane B2 (the hydrated endproduct of thromboxane A2), an important endogenous platelet activator and contractor of vascular tissue (Rao 263-75).

In addition, IRF1 (interferon regulatory factor-1) has been shown to be down-regulated in the vascularized corneas compared with the normal corneas. IRF1 serves as an activator of interferons alpha and beta (angiogenesis inhibitors) transcription. Further more, IRF1 has been shown to play roles in regulating apoptosis and tumor-suppression (Kroger et al. 1045-56).

In conclusion, the up-regulation of these genes indicates that amantadine is useful to minimize the effects due to the breakdown of the blood-retinal barrier with consequential leakage of capillaries and formation of microaneurysms.

Furthermore, amantadine up-regulates the expression of several genes that have angiogenic/angiostatic activities, specifically Sulf, IRF1, RBP1, RBP4, TIMP-3 and VEGF. HSulf-1 is a heparin-degrading endosulfatase that diminishes sulfation of cell surface. Hsulf-1 expression in ovarian cancer cell lines has been shown to reduce proliferation as well as sensitivity to induction of apoptosis (Lai et al. 23107-17). It is known that heparinases are angiogenesis inhibitors and therefore amantadine could inhibit both neovascularization and proliferation of capillary endothelial cells by increasing the gene expression of HSulf-1 (Sasisekharan et al. 1524-28).

The tissue inhibitor of metalloproteinase 3 is a very well known antiangiogenic agent. A recent study, demonstrated the ability of TIMP3 to inhibit vascular endothelial factor (VEGF)-mediated angiogenesis and identified the potential mechanism by which this occurs: TIMP3 blocks the binding of VEGF to VEGF receptor-2 and inhibits downstream signaling and angiogenesis (Qi et al. 407-15). On the other hand, VEGF is upregulated and it is known that it plays a role as an angiogenic molecule; however, it has been shown that VEGF induces IP-10 chemokine expression which is considered to be angiostatic (Lin et al. 79-82). The overall effect of amantadine on TIMP-3 and VEGF gene expression might contribute to the final antiangiogenic effect of amantadine. In addition, two retinol binding proteins are up-regulated and these proteins are the specific carrier for retinol (vitamin A alcohol) in the blood; by doing so, more retinol gets delivered to the final target tissue where in turn can explicate its antiangiogenic activity (Pal et al. 112-20).

In conclusion, the up-regulation by amantadine of the genes mentioned above, with angiogenic/angiostatic activities, would help in protecting the retina from the damage caused by aged-related macular degeneration, thereby indicating the use of amantadine to treat the above mentioned ocular diseases.

3.5 Retinitis Pigmentosa, Rod/Cone Dystrophies, Early-Onset Retinal Degeneration and Choroidal Sclerosis

Retinitis pigmentosa (RP) is the name given to a group of inherited eye diseases that affect the retina. Retinitis pigmentosa causes the degeneration of photoreceptor (rods and cones) cells or the retinal pigment epithelium (RPE) in the retina that lead to progressive visual loss. Other inherited diseases share some of the clinical symptoms of RP. Some of these conditions are complicated by other symptoms besides loss of vision. The most common of these is Usher syndrome, which causes both hearing and vision loss. Other rare syndromes include Bardet-Biedl (Laurence-Moon) syndrome, Best disease, choroideremia, gyrate-atrophy, Leber congenital amaurosis, and Stargardt disease. It should be noted that individuals who present with initial symptoms of photopsia (sensation of lights flashing), abnormal central vision, abnormal color vision, or marked asymmetry in ocular involvement may not have RP, but another retinoid cycle related retinal degeneration or retinal disease such as cone-rod dystrophy and choroidal sclerosis (information provided by Foundation Fighting Blindness at http://www.blindness.org/).

As shown in table 6, amantadine up-regulates several genes involved in the retinoid cycle such as LRAT, RPE65, RBP1/CRABP-1, RBP4, RGR, and TTR. The retinal pigment epithelium (RPE) is a monolayer simple epithelium apposed to the outer surface of the retinal photoreceptor cells. It is involved in many aspects of outer retinal metabolism that are essential to the continued maintenance of the photoreceptor cells, including many RPE-specific functions such as the retinoid visual cycle and photoreceptor outer segment disk phagocytosis and recycling. Hamel et al. (1993) characterized and cloned a unique RPE-specific microsomal protein, RPE65 that is expressed in the RPE. It has been shown that disruption of the RPE65 gene results in massive accumulation of all-trans-retinyl esters in the retinal pigment epithelium, lack of 11-cis-retinal and therefore rhodopsin, and ultimately blindness. Therefore, the effect of amantadine in increasing RPE65 gene expression in the retina would help in preventing RPE degeneration in patients affected by RP or LCA.

In addition, amantadine up-regulates LRAT, RBP1/CRABP-1, RBP4, RGR and TTR. These genes are mainly involved in the supply of all-trans-retinol to the choroidal circulation, isomerization of trans-retinal into cis-retinal and esterification of the retinol into retinyl ester in the pigment epithelium.

Amantadine increases the signal of the probeset 1389473_at which is a Rattus norvegicus transcribed sequence with similarity to protein sp:P47804 (H. sapiens) RGR_HUMAN RPE-retinal G protein-coupled receptor. A key step in the visual cycle is isomerization of all-trans retinoid to 11-cis-retinol in the RPE and RGR protein is predominantly bound to endogenous all-trans-retinal; irradiation of RGR in vitro results in stereospecific conversion of the bound all-trans isomer to 11-cis-retinal. Mutations in the human gene encoding RGR are associated with retinitis pigmentosa and choroidal sclerosis (Chen et al. 256-60).

Another important gene is lecithin: retinol acyltransferase (LRAT), which synthesizes retinyl esters by transfer of acyl moieties from phosphatidylcholine (PC). Mutations in LRAT are also associated with Leber congenital amaurosis (LCA) and early-onset retinal degeneration (Thompson et al. 123-24). Furthermore, retinoid binding proteins and transthyretin which are upregulated by amantadine have been reported to be involved in the transport of retinol in the blood to the target tissue and in the prevention of filtration of retinol in the kidney (Kuksa et al. 2959-81; Wei et al. 866-70).

In conclusion, amantadine modulates the expression of genes that are reported to be important in retinoids-cycle-related ocular diseases by improving the delivery and utilization of very important substrates for chemical reaction in the RPE and by up-regulating genes that are deficient in specific degenerative diseases such as Retinitis pigmentosa, rod/cone dystrophies, Early-onset retinal degeneration and Choroidal sclerosis.

Our findings of gene expression changes in the retina or rats treated with amantadine strongly support a benefit of amantadine in the treatment of multiple ocular diseases.

### 4. REFERENCES

Ahmed, F. et al. “Microarray analysis of changes in mRNA levels in the rat retina after experimental elevation of intraocular pressure.” Invest Opthalmol. Vis. Sci 45.4 (2004): 1247-58.
Alward, W. L. et al. “Clinical features associated with mutations in the chromosome 1 open-angle glaucoma gene (GLC1A).” N. Engl. J Med 338.15 (1998): 1022-27.
Antcliff, R. J. and J. Marshall. “The pathogenesis of edema in diabetic maculopathy.” Semin. Opthalmol. 14.4 (1999): 223-32.
Chen, P. et al. “A photic visual cycle of rhodopsin regeneration is dependent on Rgr.” Nat. Genet. 28.3 (2001): 256-60.
Chen, S. et al. “Crx, a novel Otx-like paired-homeodomain protein, binds to and transactivates photoreceptor cell-specific genes.” Neuron 19.5 (1997): 1017-30.
Ciulla, T. A., A. G. Amador, and B. Zinman. “Diabetic retinopathy and diabetic macular edema: pathophysiology, screening, and novel therapies.” Diabetes Care 26.9 (2003): 2653-64.
Endo. Symmetrel PDR Label (Endo Labs). 2000. Ref Type: Report
Fingert, J. H. et al. “Analysis of myocilin mutations in 1703 glaucoma patients from five different populations.” Hum. Mol. Genet. 8.5 (1999): 899-905.
Fiucci, G. et al. “Caveolin-1 inhibits anchorage-independent growth, anoikis and invasiveness in MCF-7 human breast cancer cells.” Oncogene 21.15 (2002): 2365-75.
Frederick, R. et al. “Modulators of the coagulation cascade: focus and recent advances in inhibitors of tissue factor, factor VIIa and their complex.” Curr. Med. Chem. 12.4 (2005): 397-417.
Freund, C. L. et al. “Cone-rod dystrophy due to mutations in a novel photoreceptor-specific homeobox gene (CRX) essential for maintenance of the photoreceptor.” Cell 91.4 (1997): 543-53.
Furukawa, T., E. M. Morrow, and C. L. Cepko. “Crx, a novel otx-like homeobox gene, shows photoreceptor-specific expression and regulates photoreceptor differentiation.” Cell 91.4 (1997): 531-41.
Furukawa, T. et al. “Retinopathy and attenuated circadian entrainment in Crx-deficient mice.” Nat. Genet. 23.4 (1999): 466-70.
Gabbay, K. H. “Hyperglycemia, polyol metabolism, and complications of diabetes mellitus.” Annu. Rev. Med 26 (1975): 521-36.
Goralski, K. B., D. D. Smyth, and D. S. Sitar. “In vivo analysis of amantadine renal clearance in the uninephrectomized rat: functional significance of in vitro bicarbonate-dependent amantadine renal tubule transport.” J Pharmacol Exp. Ther. 290.2 (1999): 496-504.
Graw, J. and J. Loster. “Developmental genetics in opthalmology.” Ophthalmic Genet. 24.1 (2003): 1-33.
Hamel, C. P. et al. “Molecular cloning and expression of RPE65, a novel retinal pigment epithelium-specific microsomal protein that is post-transcriptionally regulated in vitro.” J. Biol. Chem. 268.21 (1993): 15751-57.
Hsu, H., J. Xiong, and D. V. Goeddel. “The TNF receptor 1-associated protein TRADD signals cell death and NF-kappa B activation.” Cell 81.4 (1995): 495-504.
Ishibashi, T. et al. “cDNA microarray analysis of gene expression changes induced by dexamethasone in cultured human trabecular meshwork cells.” Invest Opthalmol. Vis. Sci 43.12 (2002): 3691-97.
Jacobson, S. G. et al. “Retinal degenerations with truncation mutations in the cone-rod homeobox (CRX) gene.” Invest Opthalmol. Vis. Sci 39.12 (1998): 2417-26.
Joza, N. et al. “Essential role of the mitochondrial apoptosis-inducing factor in programmed cell death.” Nature 410.6828 (2001): 549-54.
Klein, R., B. E. Klein, and S. E. Moss. “The Wisconsin Epidemiologic Study of Diabetic Retinopathy. XVI. The relationship of C-peptide to the incidence and progression of diabetic retinopathy.” Diabetes 44.7 (1995): 796-801.
Kroger, A. et al. “IRF-1 reverts the transformed phenotype of oncogenically transformed cells in vitro and in vivo.” Oncogene 22.7 (2003): 1045-56.
Kuksa, V et al. “Retinoid cycle in the vertebrate retina: experimental approaches and mechanisms of isomerization.” Vision Res 43.28 (2003): 2959-81.
Lai, J. et al. “Loss of HSulf-1 up-regulates heparin-binding growth factor signaling in cancer.” J. Biol. Chem. 278.25 (2003): 23107-17.
Leung, Y. F. et al. “The dual role of dexamethasone on anti-inflammation and outflow resistance demonstrated in cultured human trabecular meshwork cells.” Mol. Vis. 9 (2003): 425-39.
Li, L. et al. “Caveolin-1 maintains activated Akt in prostate cancer cells through scaffolding domain binding site interactions with and inhibition of serine/threonine protein phosphatases PP1 and PP2A.” Mol. Cell Biol. 23.24 (2003): 9389-404.
Li, X. M. et al. “Amantadine increases aromatic L-amino acid decarboxylase mRNA in PC12 cells.” J. Neurosci. Res. 53.4 (1998): 490-93.
Lin, C. S. et al. “Vascular endothelial growth factor induces IP-10 chemokine expression.” Biochem. Biophys. Res Commun. 292.1 (2002): 79-82.
Livesey, F. J. et al. “Microarray analysis of the transcriptional network controlled by the photoreceptor homeobox gene Crx.” Curr. Biol. 10.6 (2000): 301-10.
Lucetti, C. et al. “Amantadine in Huntington's disease: open-label video-blinded study.” Neurol. Sci 23 Suppl 2 (2002): S83-S84.
Mao, Y. W. et al. “Human alphaA- and alphaB-crystallins bind to Bax and Bc1-X(S) to sequester their translocation during staurosporine-induced apoptosis.” Cell Death. Differ. 11.5 (2004): 512-26.
Maren, T. H. et al. “The rates of movement of Na+, Cl−, and HCO-3 from plasma to posterior chamber: effect of acetazolamide and relation to the treatment of glaucoma” Invest Opthalmol. 1976 May; 15(5):356-64.
Marte, B. M. and J. Downward. “PKB/Akt: connecting phosphoinositide 3-kinase to cell survival and beyond.” Trends Biochem. Sci 22.9 (1997): 355-58.
Militante, J. D. and J. B. Lombardini. “Taurine: evidence of physiological function in the retina.” Nutr. Neurosci. 5.2 (2002): 75-90.
Naskar, R., C. K. Vorwerk, and E. B. Dreyer. “Concurrent downregulation of a glutamate transporter and receptor in glaucoma.” Invest Opthalmol. Vis. Sci 41.7 (2000): 1940-44.
Nicholls, D. and D. Attwell. “The release and uptake of excitatory amino acids.” Trends Pharmacol Sci 11.11 (1990): 462-68.
Pal, S. et al. “Retinoic acid selectively inhibits the vascular permeabilizing effect of VPF/VEGF, an early step in the angiogenic cascade.” Microvasc. Res 60.2 (2000): 112-20.
Qi, J. H. et al. “A novel function for tissue inhibitor of metalloproteinases-3 (TIMP3): inhibition of angiogenesis by blockage of VEGF binding to VEGF receptor-2.” Nat. Med 9.4 (2003): 407-15.
Rao, G. H. “Physiology of blood platelet activation.” Indian J Physiol Pharmacol 37.4 (1993): 263-75.
Rauen, T., J. D. Rothstein, and H. Wassle. “Differential expression of three glutamate transporter subtypes in the rat retina.” Cell Tissue Res. 286.3 (1996): 325-36.
Sasisekharan, R. et al. “Heparinase inhibits neovascularization.” Proc. Natl. Acad. Sci U.S.A 91.4 (1994): 1524-28.
Schey, K. L. et al. “Photooxidation of lens alpha-crystallin by hypericin (active ingredient in St. John's Wort).” Photochem. Photobiol. 72.2 (2000): 200-03.
Schuller-Levis, G. B. and E. Park. “Taurine: new implications for an old amino acid.” FEMS Microbiol. Lett. 226.2 (2003): 195-202.
Shen, W. Y et al. “Expression of cell adhesion molecules and vascular endothelial growth factor in experimental choroidal neovascularisation in the rat.” Br. J. Opthalmol. 82.9 (1998): 1063-71.
Snijdelaar, D. G., G. Koren, and J. Katz. “Effects of perioperative oral amantadine on postoperative pain and morphine consumption in patients after radical prostatectomy: results of a preliminary study.” Anesthesiology 100.1 (2004): 134-41.
Stone, E. M. et al. “Identification of a gene that causes primary open angle glaucoma.” Science 275.5300 (1997): 668-70.
Swain, P. K. et al. “Mutations in the cone-rod homeobox gene are associated with the cone-rod dystrophy photoreceptor degeneration.” Neuron 19.6 (1997): 1329-36.
Thompson, D. A. et al. “Mutations in the gene encoding lecithin retinol acyltransferase are associated with early-onset severe retinal dystrophy.” Nat. Genet. 28.2 (2001): 123-24.
Trencia, A. et al. “Protein kinase B/Akt binds and phosphorylates PED/PEA-15, stabilizing its antiapoptotic action.” Mol. Cell Biol. 23.13 (2003): 4511-21.
Van Hooser, J. P. et al. “Recovery of visual functions in a mouse model of Leber congenital amaurosis.” J. Biol. Chem. 277.21 (2002): 19173-82.
Verkman, A. S. “Role of aquaporin water channels in eye function.” Exp. Eye Res. 76.2 (2003): 137-43.
Wei, S. et al. “Studies on the metabolism of retinol and retinol-binding protein in transthyretin-deficient mice produced by homologous recombination.” J. Biol. Chem. 270.2 (1995): 866-70.
Xi, J. et al. “A comprehensive analysis of the expression of crystallins in mouse retina.” Mol. Vis. 9 (2003): 410-19.
Yagami, T. et al. “Effects of an endothelin B receptor agonist on secretory phospholipase A2-IIA-induced apoptosis in cortical neurons.” Neuropharmacology 48.2 (2005): 291-300.

What is claimed is:

1. A method for treating or preventing at least one ocular disorder selected from the group consisting of: loss of optic nerve fiber, breakdown of retinal vasculature, retinal damage, retinal neovascularization, retinitis pigmentosa, choroidal sclerosis, aged-related macular degeneration, and rod/cone degeneration, the method comprising:
internally administering to a patient in need thereof an effective amount of amantadine.

2. The method of claim 1, wherein the ocular disorder is at least one of:
loss of optic nerve fiber caused by at least one of: retinitis pigmentosa, choroidal sclerosis, aged-related macular degeneration, and glaucoma;
breakdown of retinal vasculature caused by diabetic retinopathy, choroidal sclerosis, aged-related macular degeneration and glaucoma;
retinal damage caused by at least one of: elevated intraocular pressure, physical injury, laser treatment, retinal ischemia, light, diabetes, and genetic predisposition; and
rod/cone degeneration caused by at least one of: light, laser treatment, and genetic predisposition.

3. The method of claim 1, wherein the administration of amantadine is at least one of: oral, parenteral, intraocular, intravitreal, intrachoroidal, and topical to the eye.

4. A method of protecting against loss of optic nerve fiber function that comprises administering an effective amount of an agent that upregulates expression of at least one of: the CRX gene, a caveolin gene, a crystallin gene, the AKT1 gene, the HSP1A gene, the SLC6A6 gene, and an Aquaporin gene.

5. The method of claim 4, wherein the agent also downregulates expression of at least one of: the PDCD8 gene and the TRADD gene.

6. The method of claim 5, wherein the agent is at least one of: adamantane and an adamantane derivative.

7. The method of claim 6, wherein the agent is amantadine.

8. A method of protecting a patient from retinal damage, such as but not limited to retinal damage resulting from elevated intra-ocular pressure (IOP), comprising:
administering an effective amount of an agent that upregulates expression of at least one of: the MYOC gene, the SLC1A3 gene, the IGFBP2 gene, the ASS gene, a crystalline gene, the SLC6A6 gene, an Aquaporin gene, and the GAD1 gene.

9. The method of claim 8, wherein the agent also downregulates expression of the ASNS gene.

10. The method of claim 8, wherein the agent is at least one of: adamantane and an adamantane derivative.

11. The method of claim 10, wherein the agent is amantadine.

12. A method of protecting a patient from at least one of: retinal neovascularization and retinal ischemia comprising:
administering an effective amount of an agent that upregulates gene expression of at least one of TIMP3, TIMP2, SULF1, IF1, RBP1, RBP4.

13. The method of claim 12, wherein the agent is at least one of: adamantane and an adamantane derivative.

14. The method of claim 12, wherein the patient is suffering from at least one of: diabetic retinopathy, diabetic macular edema, and tumorigenesis.

15. A method of identifying drug development candidates for development as retinal neuroprotective agents that comprises comparing the gene expression profile of an untreated test animal with the gene expression profile of an animal treated with a test substance, wherein the test substance is considered a candidate for development as a retinal neuroprotective agent if it is associated with the upregulation of at least one gene selected from a group consisting of CRX, crystallin genes, caveolin genes, AKT1, SLC6A6, MYOC, SLC1A3, ASS, IGFBP2, TIMP3, and Aquaporin genes.

16. The method of claim 15, wherein the effective amount is an amount effective to upregulate CRX gene expression at least about 2.65-fold.

17. The method of claim 15, wherein the effective amount is an amount effective to upregulated expression of at least one caveolin gene at least about 1.99-fold.

18. The method of claim 15, wherein the effective amount is an amount effective to upregulate expression of at least one crystallin gene at least about 3.83-fold.

19. The method of claim 15, wherein the effective amount is an amount effective to upregulate AKT1 gene expression at least about 1.69-fold.

20. The method of claim 15, wherein the effective amount is an amount effective to upregulate HSPA1A gene expression at last about 1.82-fold.

21. The method of claim 15, wherein the effective amount is an amount effective to upregulate SLC6A6 gene expression at least about 2.89-fold.

22. The method of claim 15, wherein the effective amount is an amount effective do upregulate expression of an Aquaporin gene at least about 1.68-fold.

23. The method of claim 15, wherein the effective amount is an amount effective to upregulate MYOC gene expression at least about 2.58-fold.

24. The method of claim 15, wherein the effective amount is an amount effective to upregulate SLC1A3 gene expression at least about 2.94-fold.

25. The method of claim 15, wherein the effective amount is an amount effective to upregulate IGFBP2 gene expression at least about 2.13-fold.

26. The method of claim 15, wherein the effective amount is an amount effective to upregulate ASS gene expression at least about 2.56-fold.

27. The method of claim 15, wherein the effective amount is an amount effective to upregulate TIMP3 gene expression at least about 2.34-fold.

28. A method of identifying drug development candidates for development as retinal neuroprotective agents that comprises comparing the gene expression profile of an untreated test animal with the gene expression profile of an animal treated with a test substance, wherein the test substance is considered a candidate for development as a retinal neuroprotective agent if it is associated with the downregulation of at least one gene selected from a group consisting of PDCD8, TRADD, and ASNS.

29. The method of claim 28, wherein the effective amount is an amount effective to downregulate ASNS gene expression at least about 2.12-fold.

30. The method of claim 28, wherein the effective amount is an amount effective to downregulate PDCD8 gene expression at least about 1.73-fold.

31. The method of claim 28, wherein the effective amount is an amount effective to downregulate TRADD gene expression at least about 1.75-fold.

32. A method of maintaining retinal vasculature comprising:
administering an effective amount of an agent that upregulates protein expression of at least one of: the CRX gene, a caveolin gene, a crystalline gene, the AKT1 gene, the HSP1A gene, the SLC6A6 gene, and an Aquaporin gene.

33. The method of claim 32, wherein the agent also downregulates protein expression of at least one of: the PDCD8 gene and the TRADD gene.

34. The method of claim 32, wherein the agent is at least one of: adamantane and an adamantane derivative.

35. A method of protecting a patient from retinal damage comprising:
administering an effective amount of an agent that upregulates protein expression of at least one of: the MYOC gene, the SLC1A3 gene, the IGFBP2 gene, the ASS gene, a crystallin gene, the SLC6A6 gene, and an Aquaporin gene.

36. The method of claim 35, wherein the agent also downregulates ASNS protein expression.

37. The method of claim 35, wherein the agent is at least one of: adamantane and an adamantane derivative.

38. A method of protecting a patient from retinal vascularization comprising:
administering an effective amount of an agent that upregulates protein expression of at least one of the TIMP2 gene and the TIMP3 gene.

39. The method of claim 38, wherein the agent is at least one of: adamantane and an adamantane derivative.

40. A method of identifying drug development candidates for development as retinal neuroprotective agents comprising:
comparing a protein expression profile of an untreated test animal with a protein expression profile of an animal treated with a test substance, wherein the test substance is considered a candidate for development as a retinal neuroprotective agent if it is associated with the upregulation of at least one protein selected from a group consisting of: a CRX protein, a crystallin protein, a caveolin protein, an AKT1 protein, an SLC6A6 protein, an MYOC protein, an SLC1A3 protein, an ASS protein, an IGFBP2 protein, a TIMP3 protein, and an Aquaporin protein.

41. A method of identifying drug development candidates for development as retinal neuroprotective agents comprising:
comparing a protein expression profile of an untreated test animal with a protein expression profile of an animal treated with a test substance, wherein the test substance is considered a candidate for development as a retinal neuroprotective agent if it is associated with the downregulation of at least one protein selected from a group consisting of: a PDCD8 protein, a TRADD protein, and an ASNS protein.

42. A method for obtaining regulatory approval of a therapeutic agent for treatment or prevention of an ocular disorder comprising:
providing to the governmental regulatory agency data demonstrating that the agent at least one of:
upregulates expression of at least one of: the CRX gene, a caveolin gene, a crystallin gene, the AKT1 gene, the HSP1A gene, the SLC6A6 gene, and an Aquaporin gene;
downregulates expression of at least one of: the PDCD8 gene and the TRADD gene;
upregulates expression of at least one of the MYOC gene, the SLC1A3 gene, the IGFBP2 gene, the ASS gene, a crystallin gene, the SLC6A6 gene, an Aquaporin gene, and the GAD1 gene;
downregulates expression of the ASNS gene;
upregulates expression of at least one of the TIMP3 gene, the TIMP2 gene, the SULF1 gene, and the IRF1 gene;
upregulates expression of at least one of the LRAT gene, the RBP1; CRABP-1 gene, the RBP4 gene, the RPBE65 gene, and the TTR gene; and
downregulates expression of the CA4 gene.

43. The method of claim 42, wherein the agent is for at least one of: inhibiting loss of optic nerve fiber and maintaining retinal vasculature, and wherein the data demonstrate that the agent at least one of:
upregulates expression of at least one of: the CRX gene, a caveolin gene, a crystallin gene, the AKT1 gene, the HSP1A gene, the SLC6A6 gene, and an Aquaporin gene; and
downregulates expression of at least one of: the PDCD8 gene and the TRADD gene.

44. The method of claim 42, wherein the agent is for protecting against retinal damage caused by elevated IOP and the data demonstrate that the agent at least one of:
upregulates expression of at least one of the MYOC gene, the SLC1A3 gene, the IGFBP2 gene, the ASS gene, a crystallin gene, the SLC6A6 gene, an Aquaporin gene, and the GAD1 gene; and
downregulates expression of the ASNS gene.

45. The method of claim 42, wherein the agent is for protecting a patient from retinal vascularization and the data demonstrate that the agent upregulates expression of at least one of the TIMP3 gene and the TIMP2 gene.

46. The method of claim 42, wherein the agent is for protecting against retinal damage caused at least one of: laser treatment and retinal ischemia, and wherein the data demonstrate that the agent upregulates gene expression of at least one of: the MYOC gene, the SLC1A3 gene, the IGFBP2 gene, the ASS gene, a crystallin gene, the SLC6A6 gene, an Aquaporin gene, and the GAD1 gene, downregulates ASNS gene expression, or both.

47. The method of claim 42, wherein the agent is for protecting against retinal damage caused by at least one of: light and a genetic predisposition, and wherein the data demonstrate that the agent upregulates gene expression of at least one of: the LRAT gene, the RBP1/CRABP-1 gene, the RBP4 gene, the RPE65 gene, and the TTR gene, down-regulates CA4 gene expression, or both.

48. A method of protecting a patient from at least one of: laser treatment and retinal ischemia damage comprising:
administering an effective amount of an agent that upregulates expression of at least one of: the TIMP3 gene, the TIMP2 gene, the SULF1 gene, the IRF1 gene, the RBP1 gene, the RBP4 gene, the F3 gene, the CD44 gene, the IRF1 gene, the PLA2G4A gene, and the VEGFB gene.

49. The method of claim 48, wherein the agent is at least one of: adamantane and an adamantane derivative.

50. The method of claim 49, wherein the agent is amantadine.

51. The method of claim 48, wherein the patient is suffering from at least one of: diabetic retinopathy, diabetic macular edema, diabetic macular degeneration, and ischemia retinopathy.

52. A method of protecting a patient from at least one of: light and a genetic predisposition damage comprising:
administering an effective amount of an agent that upregulates expression of at least one of: the LRAT gene, the RBP1/CRABP-1 gene, the RBP4 gene, the RPE65 gene, and the TTR gene.

53. The method of claim 52, wherein the agent is at least one of: adamantane and an adamantane derivative.

54. The method of claim 53, wherein the agent is amantadine.

55. The method of claim 52, wherein the patient is suffering from at least one of: rod/cone loss in retinitis pigmentosa, rod/cone dystrophies, and choroidal sclerosis

56. The method of claim 52, wherein the agent also downregulates CA4 gene expression.
